(19) United States
(12) Patent Application Publication
(10) Pub. No.: US 2023/0215005 A1
(43) Pub. Date: Jul. 6, 2023

(54) SYSTEMS AND METHODS FOR IMAGE MANIPULATION OF A DIGITAL STACK OF TISSUE IMAGES

(71) Applicant: Delphinus Medical Technologies, Inc., Novi, MI (US)

(72) Inventors: Mark Forchette, Novi, MI (US); John Seamans, Novi, MI (US)

(73) Assignee: Delphinus Medical Technologies, Inc., Novi, MI (US)

(21) Appl. No.: 18/184,612

(22) Filed: Mar. 15, 2023

Related U.S. Application Data
(63) Continuation of application No. PCT/US2021/050287, filed on Sep. 14, 2021.
(60) Provisional application No. 63/080,138, filed on Sep. 18, 2020.

(51) Int. Cl. G06T 7/00; G06F 3/0485

(52) U.S. Cl. CPC G06T 7/0012; G06F 3/0485; G06T 2207/30068

(57) ABSTRACT

Described herein are systems, devices, and methods for aiding a user to scroll through or otherwise manipulate a stack of medical and tissue images. A system as described herein may comprise: a foot controller, configured to detect one or more of vertical and horizontal motion of a user’s foot to control image navigation, review, positioning, and viewing, functions; a computer; and a user interface.

### CROSS-REFERENCE

This application is a continuation of PCT Application No. PCT/US2021/050287 filed Sep. 14, 2021, which claims the benefit of U.S. Provisional Application No. 63/080,138 filed Sep. 18, 2020, which application is incorporated herein by reference in its entirety.

### BACKGROUND

The present application relates to medical systems, devices, and methods, particularly for the review of imaging data by medical professionals.

Radiologists and other medical professionals often look at multiple images and screens during a typical study and often perform activities using a regular, hand-held mouse. Repetitive actions with a hand-held mouse can lead to injury such as carpal tunnel syndrome. In the case of carpal tunnel syndrome, such activities become painful, difficult, and are a significant distraction from the focus that should be given to reading stacks of images. Additionally, the use of a hand-held mouse and keyboard can only accommodate a limited number of inputs at a single time period. Therefore, improved systems, devices, and methods to replace and/or supplement some or all functions of a hand-held mouse are desirable to mitigate injury and enhance the user experience.

### SUMMARY

Disclosed herein is a method of manipulating a plurality of tissue images, comprising: providing an image analysis system, the system comprising: (i) a computing device, including a processor, a memory, and a display; (ii) one or more foot controllers operatively coupled to the computing device, a foot controller of one or more foot controllers comprising: a foot motion input receiver capable of detecting vertical movement between a first point and a second point; and (iii) the memory of the computing device storing instructions for the processor to implement a first image manipulation, the first image manipulation being a scrolling function through the plurality of tissue images in response to the foot controller being operated by a user, and cause the display to show the scrolled tissue images. The foot controller can comprise one or more horizontal motion input receivers, the one or more horizontal motion input receivers can be capable of detecting movement in a horizontal plane of motion between an inner point and an outer point, wherein the outer point is located radially away from the inner point. The image analysis system can further comprise a second image manipulation, the second image manipulation can be a search function through hanging protocols, or image layouts.

The method can further comprise determining a position of one or more horizontal motion input receivers and controlling a value of the second image manipulation as a function of the position of the one or more horizontal motion input receivers in the horizontal plane of motion. Controlling can be proportional to the movement of the one or more horizontal motion input receivers as compared to a neutral starting position. The method can further comprise proportionally controlling a value of the first image manipulation as a function of the foot motion input receiver in the vertical plane of motion.

The method can further comprise determining a position of the foot motion input receiver between the first point and the second point. The foot controller can comprise a heel switch configured to move in a vertical plane of motion between an engaged position wherein the heel switch is level with a stationary heel rest and an unengaged position wherein the heel switch is not level with the stationary heel rest.

The image analysis system can further comprise a second image manipulation, the second image manipulation can be an automatic scroll function. The method can further comprise determining a position of the heel switch and engaging or disengaging the heel switch. The foot controller can comprise one or more side switches configured to send a signal to the computer to zoom in or out when engaged. The image analysis system can further comprise a second image manipulation, the second image manipulation can be a zoom function. The method can further comprise determining a position of the one or more side switches and proportionally controlling a value of the second image manipulation as a function of engagement of the one or more side switches. The first point can be correlated with tissue images of a distal part of a tissue and the second point can be correlated with tissue images of a proximal part of said tissue. The first point can be a fully undepressed position of the foot pedal and the second point can be a fully depressed position of the foot pedal.

The digital stack of tissue images can be of a breast tissue. The digital stack of tissue images can be ultrasound images.

The foot controller can be connected to the computer wirelessly. The foot controller can be connected to the computer via USB.

The movement in the vertical plane of motion can have a first region between the first point and an intermediate point and a second region between the intermediate point and the second point. The scrolling function can be maintained at a constant minimum value of one image per movement to the intermediate point in the first region. The scrolling function can comprise a variable rate of scrolling proportional to the position of the foot pedal between the intermediate position and the second point in the second region. The variable rate of scrolling can increase in a linear manner in the second region.

Disclosed herein is a system for manipulating a plurality of tissue images, comprising: a computing device, including a processor, a memory, and a display; a foot controller operatively coupled to the computing device, the foot controller comprising a foot motion input receiver capable of detecting vertical movement between a first point and a second point; and the memory of the computing device storing instructions for the processor to implement a first image manipulation, the first image manipulation being a scrolling function through the plurality of tissue images in response to the foot controller being operated, and cause the display to show the scrolled tissue images.

The foot controller can comprise a heel switch configured to move in a vertical plane of motion between an engaged position wherein the heel switch is level with a stationary heel rest and an unengaged position wherein the heel switch is not level with the stationary heel rest. The foot controller can comprise one or more side switches comprising one or more dial mode binary switches. The one or more dial mode binary switches can be configured to be engaged into a first mode and a second mode. The first mode can be activated by a downward application of pressure on a switch of the one or more side switches and the second mode can be activated by a sideways application of pressure on a switch of the one or more side switches.

Disclosed herein is a method of manipulating a plurality of tissue images, comprising: providing an image analysis system, the system comprising: (i) a computing device, including a processor, a memory, and a display; (ii) one or more foot controllers operatively coupled to the computing device, a foot controller of one or more foot controllers comprising: a foot motion input receiver capable of detecting movement between a first point and a second point; and (iii) the memory of the computing device storing instructions for the processor to implement a first image manipulation and a second image manipulation, the first image manipulation being a scrolling function through the plurality of tissue images in response to the foot controller being operated by a user, and cause the display to show the scrolled tissue images. The foot controller can comprise one or more horizontal motion input receivers, the one or more horizontal motion input receivers can be capable of movement in a horizontal plane of motion between an inner point and an outer point, wherein the outer point is located radially away from the inner point. The second image manipulation can be a search function through hanging protocols, or image layouts.

The method can further comprise determining a horizontal position of one or more horizontal motion input receivers and controlling a value of the second image manipulation as a function of the position of the one or more horizontal motion input receivers in the horizontal plane of motion. Controlling can be proportional to the movement of the one or more horizontal motion input receivers as compared to a neutral starting position. The foot motion input receiver can be capable of detecting motion in a vertical plane of motion between the first point and the second point. The method can further comprise proportionally controlling a value of the first image manipulation as a function of the foot motion input receiver in the vertical plane of motion.

The method can further comprise determining a position of the foot motion input receiver between the first point and the second point. The foot controller can comprise a heel switch configured to move in a vertical plane of motion between an engaged position wherein the heel switch is level with a stationary heel rest and an unengaged position wherein the heel switch is not level with the stationary heel rest.

The second image manipulation can be an automatic scroll function. The method can further comprise determining a position of the heel switch and engaging or disengaging the heel switch. The foot controller can comprise one or more side switches configured to send a signal to the computer to zoom in or out when engaged. The image analysis system can further comprise a second image manipulation, the second image manipulation can be a zoom function. The method can further comprise determining a position of the one or more side switches and proportionally controlling a value of the second image manipulation as a function of engagement of the one or more side switches. The first point can be correlated with tissue images of a distal part of a tissue and the second point can be correlated with tissue images of a proximal part of said tissue. The first point can be a fully undepressed position of the foot pedal and the second point can be a fully depressed position of the foot pedal.

The digital stack of tissue images can be of a breast tissue. The digital stack of tissue images can be ultrasound images.

The foot controller can be connected to the computer wirelessly. The foot controller can be connected to the computer via USB.

The movement in the vertical plane of motion can have a first region between the first point and an intermediate point and a second region between the intermediate point and the second point. The scrolling function can be maintained at a constant minimum value of one image per movement to the intermediate point in the first region. The scrolling function can comprise a variable rate of scrolling proportional to the position of the foot pedal between the intermediate position and the second point in the second region. The variable rate of scrolling can increase in a linear manner in the second region.

Disclosed herein is a system for manipulating a plurality of tissue images, comprising: a computing device, including a processor, a memory, and a display; a foot controller operatively coupled to the computing device, the foot controller comprising a foot motion input receiver capable of detecting movement between a first point and a second point; and the memory of the computing device storing instructions for the processor to implement a first image manipulation and a second image manipulation, the first image manipulation being a scrolling function through the plurality of tissue images in response to the foot controller being operated, and cause the display to show the scrolled tissue images.

The foot controller can comprise a heel switch configured to move in a vertical plane of motion between an engaged position wherein the heel switch is level with a stationary heel rest and an unengaged position wherein the heel switch is not level with the stationary heel rest. The foot controller can comprise one or more side switches comprising one or more dial mode binary switches. The one or more dial mode binary switches can be configured to be engaged into a first mode and a second mode. The first mode can be activated by a downward application of pressure on a switch of the one or more side switches and the second mode can be activated by a sideways application of pressure on a switch of the one or more side switches. The second image manipulation can be a zoom function. The second image manipulation can be an automatic scroll function.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 is a top view of a foot controller according to embodiments described herein.

FIG. 2A is a top view of the foot controller of FIG. 1.

FIG. 2B is a side view of the foot pedal of the foot controller of FIG. 1.

FIG. 3 is a flow chart describing the process by which a foot pedal can be used manipulate a digital stack of images, according to embodiments described.

FIG. 4 is a schematic diagram of a workstation, a standard workstation interface, and a foot controller configured to manipulate a digital image stack according to embodiments described herein.

FIG. 5 is a schematic of an example processing system programable or otherwise configurable to allow presentation of a stack of images of tissue according to embodiments described herein.

FIG. 6 is a schematic of an example application provision system with one or more databases accessed by a relational database management system suitable for use with embodiments described herein.

FIG. 7 is a flow chart describing an exemplary method by which a user can manipulate a stack of images using the foot controller according to embodiments described herein.

FIG. 8 is a screenshot of an exemplary software workflow and hanging functions that can be scrolled through using a foot controller as described herein.

FIG. 9 is a screenshot of an exemplary software image review tools that can be controlled by a foot controller and/or a secondary controller in conjunction with a foot controller as described herein.

FIG. 10 is a schematic diagram of exemplary foot positions to control primary and secondary functions using a foot controller as described herein.

FIG. 11 is a flow chart describing an exemplary method by which a user can manipulate a stack of images using a foot controller as described herein.

FIG. 12A is a flow chart describing an example of foot controller inputs to control a primary scroll function.

FIG. 12B is a flow chart describing an example of foot controller inputs to control hanging protocol or image layout selection.

FIG. 12C is a flow chart describing an example of foot controller inputs to select and use a zoom function.

### DETAILED DESCRIPTION

The methods, devices, and systems described herein provide methods and tools for healthcare professionals to enable them to perform repetitive actions such scrolling through or otherwise manipulating stacks of (computerized or digital) images with a decreased risk of injury and increased productivity. The methods, devices, and systems disclosed herein, may help a user, for example, a physician to review and manipulate a stack of images with the use of a foot controller

Although the examples of the disclosure are illustrated on specific embodiments of a foot controller, embodiments described herein can be implemented for any other alternative foot controller system. Two foot controllers can be used simultaneously, for example one for a right foot of a user and one for a left foot of a user. Devices and methods of use as disclosed herein may be used to review images from a variety of imaging modality such as magnetic resonance imaging (MRI) or computed tomography (CT) or positron emission tomography (PET) or a combination of imaging modalities. Devices and methods of use as disclosed herein may be used to review images of a number of biological tissues. A biological tissue may comprise an organ or tissue of a patient or subject. The methods and systems described herein can further be implemented on the images from any organ or tissue of the body. The organ or tissue may comprise for example: a muscle, a tendon, a ligament, a mouth, a tongue, a pharynx, an esophagus, a stomach, an intestine, an anus, a mammary gland, a liver, a gallbladder, a pancreas, a nose, a larynx, a trachea, lungs, a kidneys, a bladder, a urethra, a uterus, a vagina, an ovary, a testicle, a prostate, a heart, an artery, a vein, a spleen, a gland, a brain, a spinal cord, a nerve, etc, to name a few.

Other biological tissue may comprise a body part, such as a brain, a foot, a hand, a knee, an ankle, an abdomen, muscles, a tendon, a ligament, a mouth, a tongue, a pharynx, an esophagus, a stomach, an intestine, an anus, a liver, a gallbladder, a pancreas, a nose, a larynx, a trachea, lungs, a kidney, a bladder, a urethra, a uterus, a vagina, an ovary, a breast, a testes, a prostate, a heart, an artery, a vein, a spleen, a gland, a spinal cord, a nerve, or any other body part. A body part may be operatively attached to or contained within a living human being. In some embodiments, the body part comprises muscular tissue, fatty tissue, bone, etc. The body part may be a human body part. The body part may be a body part of a non-human animal, such as a body part of a mouse, cat, dog, bird, pig, sheep, bovine, horse, or non-human primate.

The terminology used herein is for the purpose of describing particular embodiments only and is not intended to be limiting of the claims. As used in the description of the embodiments and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range.

As used herein, the terms “comprises,” “comprising,” or any other variation thereof, are intended to cover a nonexclusive inclusion, such that a process, method, article, or apparatus that comprises a list of elements does not include only those elements but may include other elements not expressly listed or inherent to such process, method, article, or apparatus.

As used herein, the terms “subject” and “patient” are used interchangeably. As used herein, the terms “subject” and “subjects” refers to an animal (e.g., birds, reptiles, and mammals), a mammal including a primate (e.g., a monkey, chimpanzee, and a human) and a non-primate (e.g., a camel, donkey, zebra, cow, pig, horse, cat, dog, rat, and mouse). In certain embodiments, the mammal is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old.

As used herein, the term “user” refers to a healthcare professional or any individual using the methods and systems of the present disclosure including but not limited to physicians such as radiologists.

Unless otherwise defined, all technical terms used herein have the same meaning as commonly understood by one of ordinary skill in the art to which this invention belongs.

Reference will now be made in detail to various embodiments, examples of which are illustrated in the accompanying drawings. In the following detailed description, numerous specific details are set forth in order to provide a thorough understanding of the present disclosure and the described embodiments. However, the embodiments of the present disclosure are optionally practiced without these specific details. In other instances, well-known methods, procedures, components, and circuits have not been described in detail so as not to unnecessarily obscure aspects of the embodiments. In the drawings, like reference numbers designate like or similar steps or components.

### Foot Controller

The system for aiding a user to manipulate a stack of images may comprise a foot controller. As an alternative or addition to a regular hand controller such as a keyboard and mouse, the systems for aiding a user to manipulate a stack of images and methods disclosed herein may facilitate a user’s productivity in manipulating a stack of images. For example, the system may alleviate the strain of repetitive hand motions that could lead to injury such as carpal tunnel syndrome and/or provide additional functionality to supplement a typical user interface such as a keyboard, mouse, touch screen display, and/or voice control.

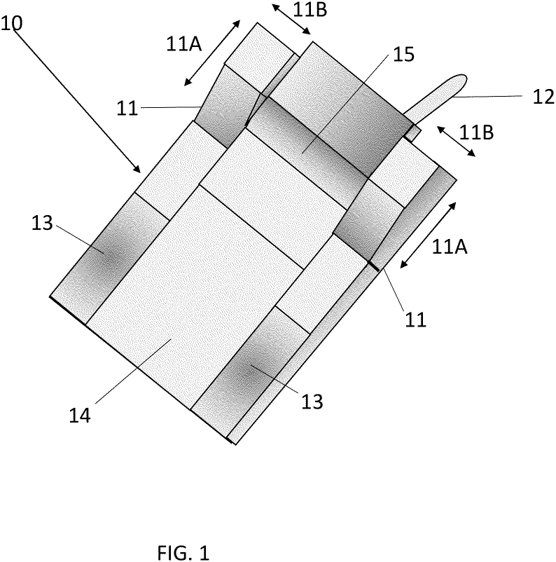

FIG. 1 shows an example of a foot controller of the system for aiding a user to sort through or otherwise manipulate a stack of medical, tissue, or other images according to some embodiments. FIG. 1 shows a front, perspective view of a foot controller 10. Foot controller 10 can have a body with a base that supports foot controller on the floor. The body may include a foot pedal 15, a heel rest 14, a left toe switch 11A, a right toe switch 11A, a left heel switch 13, a right heel switch 13, and optionally a wire or cable 12 (e.g., a USB cable) to connect to a computer system. Alternatively or in combination, the foot controller 10 may communicate wirelessly with the computer system, such as through Bluetooth or other wireless connectivity protocol.

The foot controller can be configured for a right foot or a left foot. The foot controller can be adjustable so as to accommodate different foot widths and lengths. The pedal and side buttons of a foot controller can be configured to slide on two perpendicular tracks to accommodate different foot widths and lengths. The side buttons and pedal can be configured to lock into a position in order to accommodate the length and width of an operator’s foot.

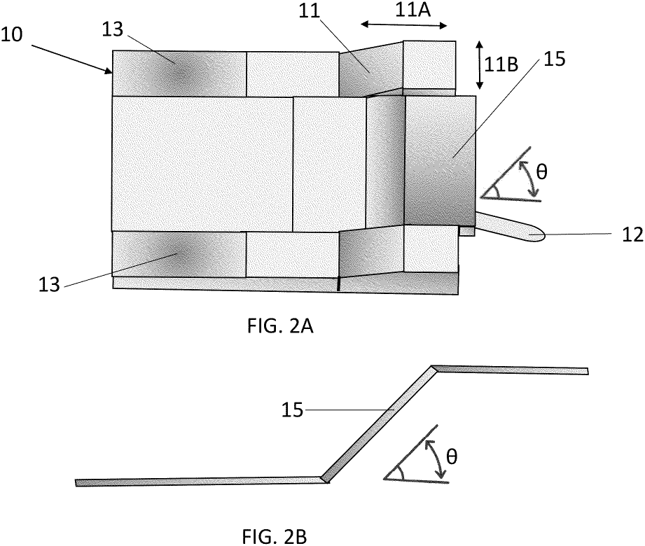

FIG. 2A is a top view of the foot controller 10. The foot controller 10 can be rotationally coupled to the body. The foot pedal 15 of the foot controller 10 may be depressed using the upper portion of the user’s foot to move from a fully undepressed positions to a fully depressed position in which the foot controller lies in generally the same plane as the heel rest (or any level of depression therebetween). The foot pedal 15 of the foot controller 10 may be configured to move in a substantially vertical manner, the movement may be similar to a gas pedal of a car. The foot pedal 15 of the foot controller 10 may be configured to move with a rotation axis at the lower or upper portion of the foot which may revert to an initial position when released, the movement may be similar to a Wah pedal. The foot pedal 15 of the foot controller 10 may be configured to move in a substantially horizontal manner in which the foot pedal 15 moves forward and backward with a rotation axis in the middle of the foot which may be configured to revert to an initial position when released. Depression of the foot pedal 15 may allow a user to scroll linearly through an image stack. For example, depression of the foot pedal 15 may allow a user to scroll linearly through an image stack of a breast tissue from nipple to chest wall. In some embodiments, the degree to which the foot pedal 15 is depressed may control the speed of the scrolling. For instance, the image scrolling may increase in rate the greater degree the foot pedal 15 is depressed. FIG. 2B is a side view of a foot pedal 15 of a foot controller 10 wherein linear movement described by angle theta is used to control scrolling through images, for example from the most anterior location (such as a nipple) at the initial pedal angle to a posterior location (such as a chest wall) at where the pedal angle theta is at 0 degrees The plane of the heel rest 14 may be disposed at an angle relative to the plane of the base to increase a user’s comfort. Alternatively, the plane of the heel rest 14 may be parallel to the plane of the base. The foot controller may be used to provide proportional control of functions of image searching such as moving through hanging protocols, linear movement to go from a distal end of a tissue to a proximal end, auto scroll, and zoom.

The foot controller 10 may accommodate multiple image manipulation and/or system control modes. The left toe switch 11 may be a dual mode binary switch. The first mode of the switch, 11A, may be actuated when a user presses downward on the switch with their toe. This first mode is referred to herein as a left vertical switch, 11A. The first mode may control a zoom function. The first mode of the left toe switch may be activated by the user to zoom in proportionally to the downward motion of the user’s toe on the left toe switch. In some embodiments, the degree to which the foot pedal 15 is depressed may control speed of the zooming in or magnification. For instance, the zooming or magnification may increase in rate the greater degree the foot pedal 15 is depressed. Alternatively, the first mode of the left two switch, 11A, may be activated by the user to zoom out proportionally to the downward motion of the user’s toe on the left toe switch. The second mode of the left toe switch, 11B, may be actuated when a user presses in a generally outward, horizontal direction on the switch with a side of their foot. The second mode is referred to herein as a left horizontal switch 11B. The second mode of the left toe switch may allow a user to move forward through a menu of hanging or other protocols. Alternatively, the second mode of the left toe switch may allow a user to move backward through a menu of hanging protocols. A switch can be a momentary actuation type switch that provides tactile feedback to the user.

The right toe switch 11 can likewise be a dial mode binary switch. The first mode of the switch, 11A, can be actuated when a user pressed downward on the switch with their toe. The first mode can be referred to herein as a right vertical switch, 11A. The first mode may control a zoom function. The first mode of the right toe switch 11 may be activated by the user to zoom in proportionally to the downward motion of the user’s toe on the right toe switch 11. In some embodiments, the degree to which the foot pedal 15 is depressed may control speed of the zooming out. For instance, the zooming or magnification may increase in rate proportional to the greater degree the foot pedal 15 is depressed. Alternatively, the first mode of the right toe switch 11 may be activated by the user to zoom out proportionally to the downward motion of the user’s toe on the right toe switch 11. The second mode, 11B, can be actuated when a user presses in a generally outward, horizontal direction on the switch with the side of his or her foot. The second mode can be referred to as a right horizontal switch 11B. The second mode of the right toe switch may allow a user to move forward through a menu of hanging protocols. Alternatively, the second mode of the right toe switch may allow a user to move backward through a menu of hanging protocols. The switch can be a momentary actuation type switch that provides tactile feedback to the user.

The heel switch 13 can be a momentary actuation type that allows a user to engage auto-scroll through a stack of images.

The foot controller can be configured to provide feedback to a user when a switch is engaged, for example by vibration, audio, or visual signals. For example, when the user scrolls through an image stack and reaches the end of the stack, the pedal may vibrate.

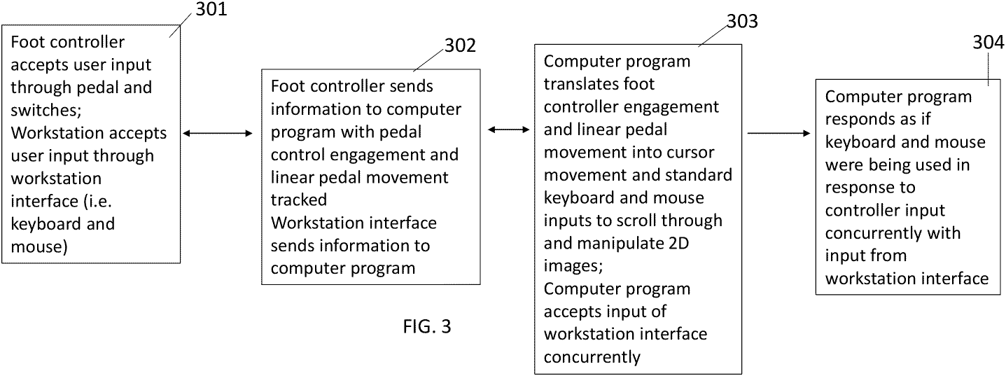

FIG. 3 is a flow chart describing a process 300 by which the foot pedal can be used to manipulate a stack of images concurrently with a workstation interface such as a keyboard and mouse. In 301, the foot controller can accept a user input through a foot pedal and switches concurrently with an input from a workstation interface (i.e., a keyboard and mouse). In 302, the foot controller can then send information to the computer program which tracks the pedal engagement and linear movement as well as any input from the workstation interface. In 303, the computer program can then translate the tracked input from the foot controller into cursor movements and standard keyboard and mouse inputs to scroll through and manipulate the stack of images while concurrently accepting input from the workstation interface. In 304, the computer program can then respond to the input from the foot controller and the workstation interface.

Although the above operations show a method 300 for using a foot controller, in accordance with some embodiments, a person of ordinary skill in the art will recognize many variations based on the teachings described herein. The steps may be completed in any order. Steps may be added or deleted. Some of the steps may comprise sub-steps. Many of the steps may be repeated as often as beneficial to the method.

The foot controller can comprise a sensor capable of detection both horizontal and vertical motion of a user’s foot. The sensor can be a clip on sensor. The sensor can be a clip on sensor configured to attach to a user’s shoe. The sensor can comprise a gyroscope to detect horizontal and vertical foot motion relative to a starting point. The sensor can comprise an accelerometer to detect a rate of motion. The foot controller can comprise a capacitor. The foot controller can comprise a light or laser based sensor. The foot controller can include a mat which defines an active area where the sensor’s inputs are received to prevent activation of controls when the foot is not on the mat. The position of a user’s foot and rate of motion of a user’s foot can be used to proportionally control functions such as scrolling through an image volume, selecting different hanging protocols or image layouts, and zooming in/out. For example, a user may double tap their foot in a quick succession to activate an interactive zoom function. A user may select a function such as automatic image scrolling by tapping their foot, i.e. a single tap to enter a scroll speed setting wherein moving a user’s foot to the right of a starting point increases the speed and moving a user’s foot to the left of the starting point decreases the speed. The heel of the user’s foot may be substantially stationary. The user may deselect the function by tapping their foot a second time.

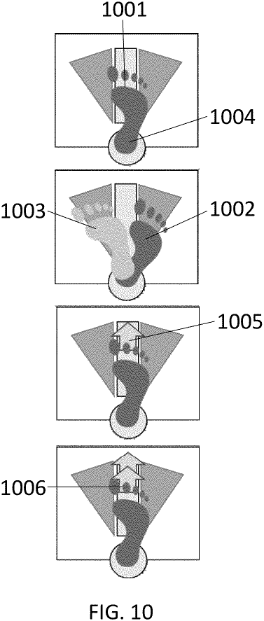

As can be seen in FIG. 10, the foot position of the user can activate primary actions such as scrolling. A user may initiate a first primary action by moving the ball of their foot to a first position on the right 1002 or left 1003 of a neutral position 1001 while maintaining the heel of their foot in a single position 1004. Primary actions include scroll rate and direction. Scroll can may be a standard rate or a fast rate. Scroll can increase or decrease in speed in proportion to the distance the user moves their foot left or right of the neutral position, keeping the heel of their foot substantially stationary. Scroll direction can be anterior or posterior through a stack of images. A user may initiate secondary actions by vertically tapping their foot one 1005 or more times 1006. Secondary actions include tool selection, zoom, hanging protocol / image layout selection, etc. For example, after hanging protocol selection mode is initiated via vertical foot tap, the next or previous hanging protocol / image layout can be selected by moving the ball of the foot to the right 1002 or left 1003, respectively. Similarly, after it is selected by a double foot tap, zoom in or out are achieved by moving the ball of the foot to the right 1002 or left 1003, respectively.

A user can be guided visually by a diagram to aid in determining a foot position. The diagram can be on the floor of a user’s workstation. The diagram may be a mat on the floor. The diagram may be a light projection on the floor. The diagram may be integrated into the user interface such as on a computer screen. The user may be guided by haptic or auditory feedback from the foot controller. The haptic feedback can be a vibration. The auditory feedback can be a beep or other sound. The user may be guided by a visual aid on a computer screen of a user interface. The foot controller may be used in conjunction with a foot support to help the user maintain a foot position over a period of time, such as a foot pedal structure that allows a user to move their foot in a controlled manner in a vertical plane. The foot controller may be used in conjunction with a heel switch.

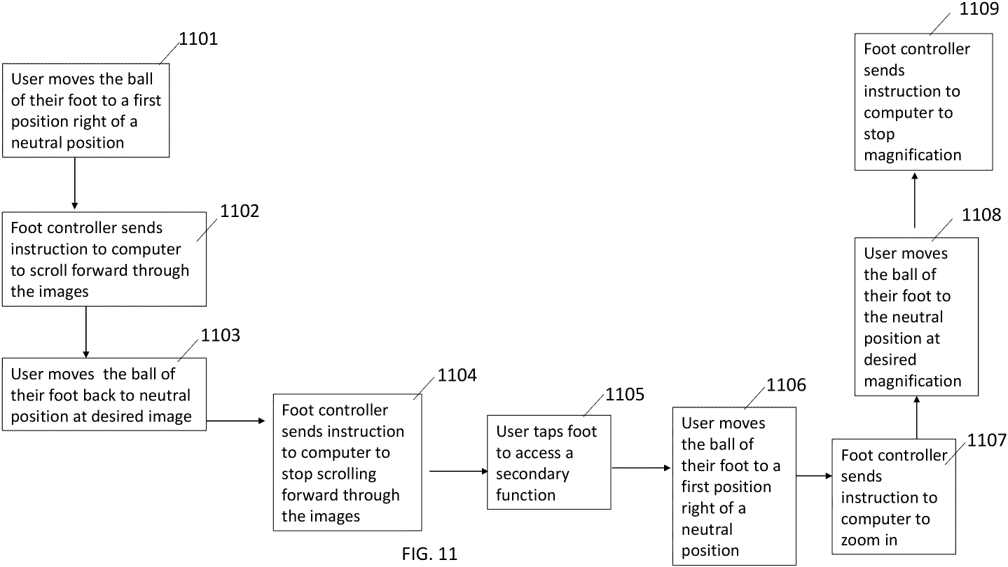

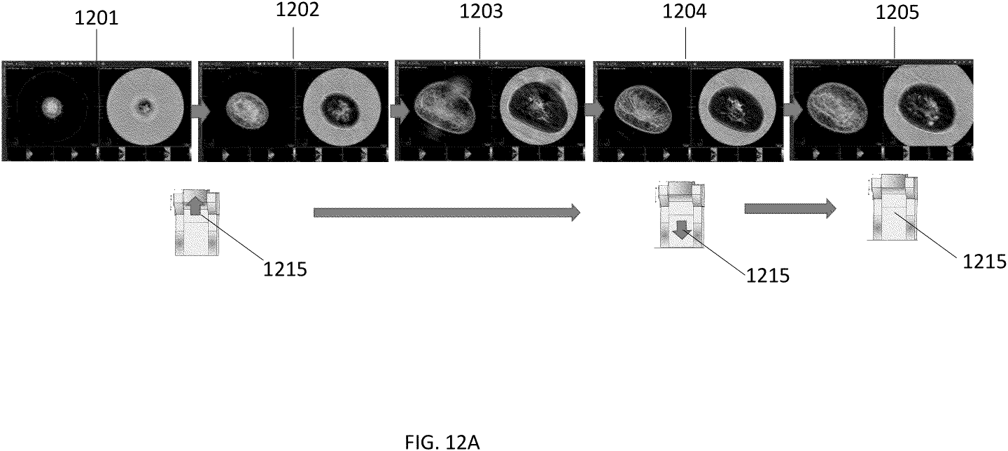

Referring now to FIG. 11, a flow chart of exemplary functions of the foot controller for use in a method 1100 is shown. In 1101, a user may engage the foot pedal of the foot controller by moving the ball of their foot to a first position right of a neutral position. In 1102, the foot controller may send a signal to the computer program, which interprets the signal as a command to scroll forward through the image stack. In 1103, the user may then move the ball of their foot back to a neutral position when they reach the desired image. In 1104, the foot controller may then send a signal to the computer program, which interprets the signal as a command to stop scrolling. In 1105, to zoom in on the image, the user may then tap their foot to access a secondary zoom function. In 1106, the user moves the ball of their foot to a first position right of a neutral position to zoom in on the image. In 1107, responding to the input from the user, the foot controller may send a signal to the computer program, which interprets the signal as instruction to zoom in on the image. In 1108, to stop the magnification, the user may then move the ball of their foot to a neutral position at the desired magnification. In 1109, responding to input from the user, the foot controller may send a signal to the computer program, which interprets the signal as instruction to stop magnification of the image. The user may then zoom out of the image by moving the ball of their foot to a position left of the neutral position. The user may access hanging protocol selection by double tapping the ball of their foot in a quick succession of vertical up and down motions. The user can then move the ball of their foot to a position right or left of the neutral position to move to next or previous hanging protocol / image layout, respectively. To return to primary functions, the user can tap the ball of their foot.

The foot controller may be configured to transmit a signal to the user interface computer when within a specified distance from the user interface computer. The foot controller may be configured to stop transmitting a signal if a heel position of a user is moved. In some instances, the foot controller can be used in conjunction with a heel switch and the foot controller can be configured to stop transmitting a signal if the foot controller is moved beyond a proximity to the heel switch. The foot controller may be activated using the standard workstation interface (e.g. mouse or keyboard). The foot controller may be deactivated using the standard workstation interface (e.g. mouse or keyboard).

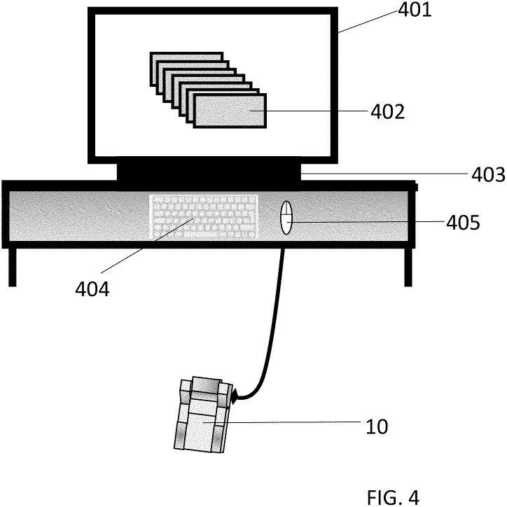

FIG. 4 is a schematic diagram of an exemplary workstation 400 comprising one or more computer screens 401 to display a stack of tissue images 402 wherein the user can manipulate the state of tissue images using a foot controller 10 alone or in combination with a workstation interface such as a keyboard 404 and a mouse 405, along with the requisite image manipulation software. Image manipulation software can include Hologic SecurView, MIM Software, OsiriX, Three Palm Workstation One, Vital Images Vitrea, Siemens Syngo, GE Advantage Workstation, etc. from the foot controller 10, keyboard 404, and mouse 405 can be processed by a computer processor 403 running a program configured to translate the inputs from the workstation interface (i.e., keyboard 404 and mouse 405) and the foot controller 10 to manipulate the stack of images.

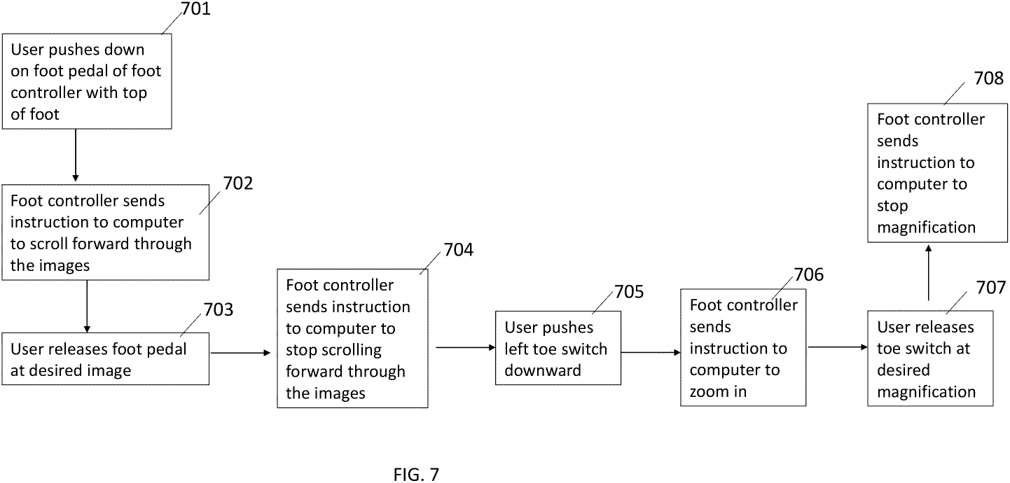

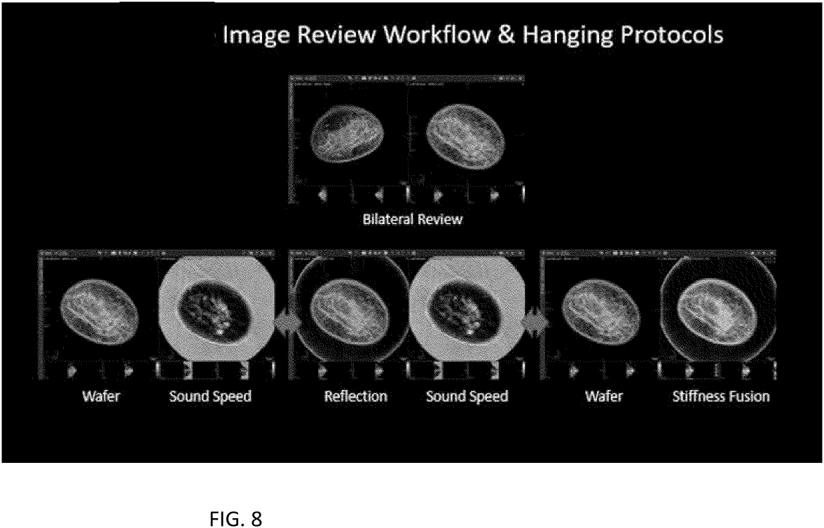

Referring now to FIG. 7, a flow chart of exemplary functions of the foot controller for use in a method 700 is shown. In 701, a user may engage the foot pedal of the foot controller by pressing down on the foot pedal with their foot. In 702, the foot controller may send a signal to the computer program, which interprets the signal as a command to scroll forward through the image stack. In 703, the user may then remove pressure on the foot pedal by lifting their foot when they reach the desired image. In 704, the foot controller may then send a signal to the computer program, which interprets the signal as a command to stop scrolling. In 705, to zoom in on the image, the user may then push the left toe switch downward with their foot, using the heel rest of the foot controller as a pivot point for their foot. In 706, responding to the input from the user, the foot controller may send a signal to the computer program, which interprets the signal as instruction to zoom in on the image. In 707, to stop the magnification, the user may then release the toe switch at the desired magnification. In 708, responding to input from the user, the foot controller may send a signal to the compute program, which interprets the signal as instruction to stop magnification of the image. The user may then zoom out of the image by pressing down on the right toe switch, or scroll through hanging functions by sliding the left and right toe switches to move left or right through the functions, respectively.

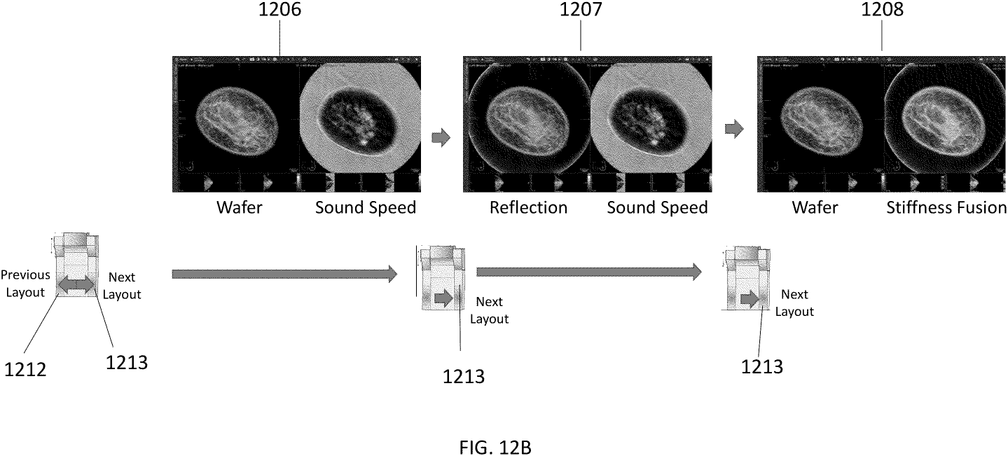

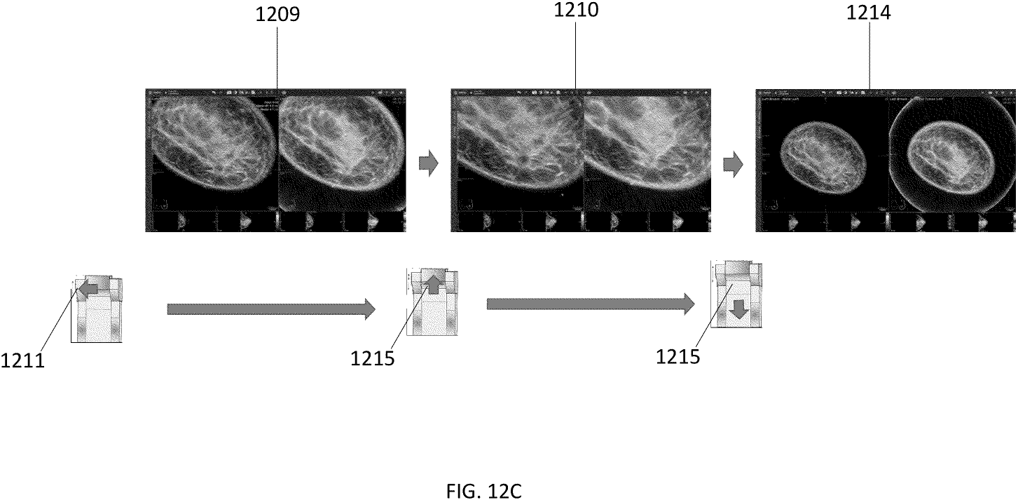

Referring now to FIG. 12A, a flow chart showing an exemplary method of using a pedal of a foot controller 1215 to perform a primary function of scrolling through a stack of images. A user may scroll through an image volume from a posterior location of the volume 1201 to an anterior location 1202, to the end of an image volume 1203, by pushing a pedal 1215 of a foot controller in a first direction. A user may scroll in a second, reverse direction, through an image volume from an anterior location of the volume 1204 to a desired location of the image volume 1205 by pushing a pedal 1215 of a foot controller in a second direction. A user may remain at a desired location of the image volume 1205 by removing pressure from the pedal 1215.

Referring now to FIG. 12B, a flow chart showing an exemplary method of using a pedal of a foot controller 1215 to select hanging protocols or image layout selections. A user may view a first image layout 1206 using a first heel switch of a foot controller 1213. A user may view a second image 1207 layout using a first heel switch of a foot controller 1213. A user may then view a third image layout 1208 using a first heel switch of a foot controller 1213. A second heel switch 1212 may be used to view a previous layout.

Referring now to FIG. 12C, a flow chart showing an exemplary method of using a switch of a foot controller 1211 to select a zoom function and use a pedal of a foot controller 1215 to zoom in or out on an image. A user may select a zoom function by applying horizontal pressure to a switch of a foot controller 1211. A user may zoom in on an image 1209 to a desired magnification of the image 1210 by pushing a pedal 1215 of a foot controller in a first direction. A user may zoom out from an image 1210 to a desired magnification of the image 1214 by pushing a pedal 1215 of a foot controller in a second direction. A user may remain at a desired magnification of the image 1214 by removing pressure from the pedal 1215.

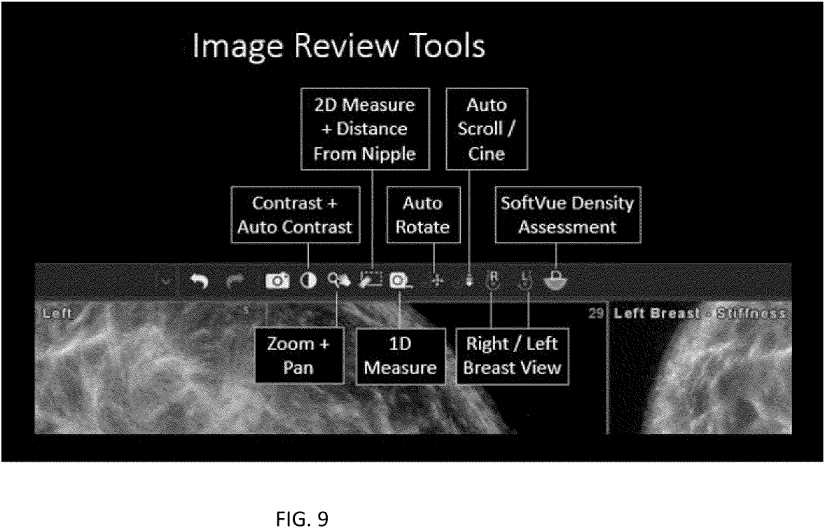

FIG. 8 is a screenshot of hanging and workflow protocols in one exemplary application, SoftVue. The foot controller as described herein can be used to scroll through and apply workflow and hanging protocols such as Bilateral Review, Wafer- Sound Speed, Reflection - Sound Speed, and Wafer- Stiffness Fusion. Similar protocols in alternative image review software applications, for example Hologic SecurView, MIM Software, OsiriX, Three Palm Workstation One, Vital Images Vitrea, Siemens Syngo, GE Advantage Workstation, etc are contemplated.

FIG. 9 is a screenshot of secondary functions, i.e., image review tools in one exemplary application, SoftVue, which can be controlled by a secondary controller in conjunction with a foot controller as described herein. The secondary controller can be a second foot controller or a hand controller. Image review tools can include contrast adjustment, zoom, 1D measurement, 2D measurement, auto rotation, auto-scroll, image view selection, and density assessment. Similar tools in alternative image review software applications, for example Hologic SecurView, MMviewer, OsiriX MD, and Three Palm Workstation One, are contemplated.

In some embodiments, ultrasound images, for example, ultrasound tomography images, are used in the methods and systems of the present disclosure. Tissue images may be obtained by emitting acoustic waveforms and detecting a set of acoustic signals may be performed with an ultrasound tomographic scanner, for example, using methods similar to those described in U.S. Pat. Nos. 6,385,474; 6,728,567; 8,663,113; 8,876,716; and 9,113,835; and U.S. Publication Nos. 2013/0041261 and 2013/0204136, which are each incorporated by reference in their entirety. However, any suitable ultrasound device or scanner may be used. A sound speed rendering may be generated from a waveform sound speed method. Such a method may comprise generating an initial sound speed rendering in response to simulated waveforms according to a travel time tomography algorithm. The initial sound speed rendering may be iteratively optimized until ray artifacts are reduced to a pre-determined a threshold for each of a plurality of sound frequency components. The initial method rendering may be iteratively adjusted until the obtained model is good enough as a starting model for the waveform sound speed method to converge to the true model. Such a method may comprise the method described in U.S. App. No. 14/817,470, which is incorporated herein in its entirety by reference.

The images may be obtained using a scanner table. The scanner table may comprise an embodiment, variation, or example of the patient interface system described in any of the references incorporated herein and additionally or alternatively in U.S. Application Ser. No. 14/208,181, entitled “Patient Interface System”, U.S. Application Ser. No. 14/811,316 entitled “System for Providing Scanning Medium”, or P.C.T. International Pat. App. Pub. No. WO2017139389 entitled “System for Shaping and Positioning a Tissue Body”, which are each hereby incorporated by reference in their entirety. However, an ultrasound system may additionally or alternatively comprise or be coupled with any other suitable patient interface system.

In some embodiments, systems and methods of the present disclosure may be used to aid a user to generate or manipulate an enhanced reflection image. An enhanced reflection image may comprise an embodiment, variation, or example of the system and method for generating an enhanced image of a volume of tissue described in commonly assigned applications: U.S. Pat. App. No. 15/829,748 and P.C.T. App. No. PCT/US2017/064350, which are each incorporated herein by reference in their entirety. Briefly, systems and methods of the cited reference may combine a reflection image generated from detection a reflected signal from a volume of tissue and a speed image to generate an enhanced reflection image. The second reflection image may be generated from a gradient of a sound speed image, and the two reflection images may be combined as described in the incorporated references.

In some embodiments, systems and methods of the present disclosure may be used in combination with one or more images or renderings from a human or other animal. As such, in one variation, images used in combination with the system may be reviewed by a user to characterize the tissue to facilitate diagnoses of cancer, assess its type, and determine its extent (e.g., to determine whether a mass in the tissue may be surgically removable), or to assess risk of cancer development (e.g., measuring breast tissue density). A stiffness rendering may be generated based upon one or more of a sound speed rendering and an acoustic attenuation rendering. A stiffness rendering may be generated based upon a combination of a sound speed rendering and an acoustic attenuation rendering. A stiffness rendering may comprise one or more of: a distribution of colors corresponding to different stiffness values or ranges of stiffness values, a distribution of patterns corresponding to different stiffness values or ranges of stiffness values, a distribution of shading (e.g., intensity) corresponding to different stiffness values or ranges of stiffness values, a distribution of a saturation parameter corresponding to different stiffness values or ranges of stiffness values and any other suitable method of representing a distribution of a parameter within a volume of tissue. Methods of generating a stiffness rendering may comprise methods described in U.S. App. No. 14/703,746, which is incorporated herein in its entirety by reference.

One or more image characterization parameters may be extracted from the one or images using the systems and methods disclosed herein. Image characterization parameters may be determined by the observations of a skilled user, e.g. a clinician. Image characterization parameters may be determined wholly by or with the aid of a computer and the systems and methods described herein. The one or more image characterization parameters may comprise an embodiment, variation, or example of the image characterization parameters (e.g. prognostic parameters) in P.C.T. App. No. PCT/US2019/029592, which is incorporated herein by reference in its entirety. An example of determining image characterization parameters wholly by or with the aid of a computer is provided in P.C.T. App. No. PCT/US2019/029592, which is incorporated herein by reference in its entirety.

A parameter of a set of parameters of an image or data set may comprise an average value of a sound propagation metric within a tumor, a kurtosis value within a tumor, a difference between a kurtosis values within a tumor and a kurtosis value within a peri-tumor, a standard deviation of a grayscale within a tumor, a gradient of a grayscale image within a tumor, a standard deviation of a gradient within a peri-tumor, a skewness of a gradient within a peri-tumor, a kurtosis of a corrected attenuation within a peri-tumor, a corrected attenuation of an energy within a tumor, a contrast of a grayscale of an image within a peri-tumor, a homogeneity of a grayscale of an image within a peri-tumor, or a difference in contrast of a grayscale within a tumor and within a peri-tumor.

A parameter of a set of parameters of an image or data set may comprise order statistics (i.e. mean, variance, skewness, kurtosis, contrast, noise level, signal to noise ratio (SNR), etc.) of the underlying acoustic parameters (the raw pixel value of each image) or the gray/color scale counter parts.

In some cases, the texture of the images can be assessed by using order statistics of histograms characterizing the value of grayscale distributions. Grayscale is a collection of the range of monochromatic (gray) shades. Grayscale may range from white to black. In terms of luminescence, grayscale may range from bright to dark. In some embodiments, features herein include texture features such as first order histogram features. In some embodiments, the features include higher order features which further characterize texture such as gray level co-occurrence matrices (GLCM) and their respective scalar features (energy, entropy, etc.) In some embodiments, the co-occurrence matrix herein is method which compares the intensity of a pixel with its local neighborhood. In some embodiments, the co-occurrence matrix can examine the number of times a particular value (in grey scale) co-occurs with another in some defined spatial relationship.

In some cases, the images may be assessed using parameters derived from colorscale images. Colorscale may relate to renderings of ultrasound images such as stiffness images that can be shown in color. These images may be representative of stiffness properties of volume of tissue such as breast tissue. The color map may range from black to red. In some embodiments, the color map may range from blue to red. Other color ranges may be defined for the volume of tissue representing a range of stiffness parameters. The stiffer the tissue, the closer the color may be to the red end of the color range. The color black or blue may be indicative of no stiffness or absence of stiffness. In some cases, these parameters may be derived from order statistics of histograms characterizing the value of grayscale distributions.

A parameter of a set of parameters in an image or data set may comprise a “lucent halo.” In some sound speed images, dark rings (lucent halos) may surround the volume of tissue such as breast tissue. Lucent halo may be observed in some fibroadenomas or cysts. Lucent halos may be indicative of benign process.

A parameter of a set of parameters an image or data set may comprise a kurtosis value. A kurtosis value can describe or represent the sharpness of a peak of a frequency-distribution curve. In some cases, kurtosis can be calculated as a kurtosis of a gradient of an image such as a grayscale image. Kurtosis can be determined for any type of image, for example, a corrected attenuation image, an enhanced reflection image, a compounded enhanced reflection image, a sound speed image, etc.

A parameter of a set of parameters an image or data set may comprise a contrast. A contrast can be a measure of a difference in signal within a ROI such as a tumor or a peri-tumor.

A parameter of a set of parameters an image or data set may comprise a homogeneity value. A homogeneity value can be a measure of variation within a ROI such as a tumor or a peri-tumor.

A parameter of a set of parameters an image or data set may comprise at least one texture metric of a ROI. A texture metric can comprise at least one of an edgeness, a grey level co-occurrence matrix, and a Law’s texture map.

A parameter of a set of parameters an image or data set may comprise a parameter of a wavelet of an image. In some cases, a wavelet of an image can represent an image. In some cases, a wavelet can be employed in the analysis of an image. Examples of wavelets can include a continuous wavelet transform of an image or a discrete wavelet transform of an image.

A parameter of a set of parameters an image or data set may comprise a standard deviation of an eroded grayscale image within a tumor, an average of an eroded grayscale image within a tumor, a standard deviation of an eroded grayscale image within a peri-tumor, a first order entropy of a gradient within a tumor, a first order mean of a gradient within a tumor, a difference between a first order entropy within a tumor and a first order entropy within a peri-tumor, a contrast within a tumor, a correlation within a tumor, a difference in contrast between a tumor and a peri-tumor, or a difference in homogeneity between a tumor and a peri-tumor.

A parameter of a set of parameters an image or data set may comprise one or more of the margin boundary score, the mean enhanced reflection, the relative mean of the enhanced reflection interior and exterior to the ROI, the standard deviation of the enhanced reflection, the mean sound speed, the relative mean sound speed interior and exterior to the ROI, the standard deviation of the sound speed, the mean attenuation, the standard deviation of the attenuation, the mean of the attenuation corrected for the margin boundary score, and the standard deviation of the attenuation corrected for the margin boundary score.

A parameter of a set of parameters an image or data set may comprise one or more of an irregularity of a margin, an average of sound speed values within a tumor, an average attenuation value within a peri-tumor, a contrast texture property of reflection within a peri-tumor, a difference between an average reflection value within a tumor and an average reflection value within a peri-tumor, a contrast texture property of a reflection within a tumor, a first order standard deviation of a sound speed value within a tumor, an average of a reflection value within a tumor, an average of a reflection value within a peri-tumor, a first order average of a reflection value within tumor, a difference between a homogeneity texture property of a reflection within a tumor and within a peri-tumor, a first order average of a sound speed value within a peri-tumor difference between a contrast texture property of an attenuation within a tumor and a contrast texture property of an attenuation within a peri-tumor, a standard deviation of a wavelet detail coefficient of a sound speed margin, a standard deviation of a wavelet detail coefficient of a reflection margin, a histogram of entropy of a wavelet detail coefficient of a reflection margin, a local minimum standard deviation of a wavelet detail coefficient of reflection margin, or a maximum of a standard deviation of a crisp contrast.

As described herein above, each image may comprise a portion of a rendering. For example, a rendering may be formed from one or more “stacks” of 2D images corresponding to a series of “slices” of the volume of tissue for each measured acoustomechanical parameter at each step in a scan of the volume of tissue. Each slice may comprise an image or layer of the rendering. Each layer, subset of layers, classification of layers, and/or ROI may have one or many associated parameters, for example, any type of parameter associated with image characteristics as described herein.

A parameter of a set of parameters an image or data set may comprise volumetric parameters. A volumetric parameter may be derived from a plurality of image positions along an anterior-exterior axis of tissue. In some cases, a volumetric parameter may be a qualitative volumetric parameter. In some cases, a volumetric parameter may be a quantitative volumetric parameter.

In an example of a qualitative volumetric parameter, a user may indicate whether or not a region of interest “flows” from layer to layer of the stack of 3D images. The parameter “flows” may be used to distinguish dense tissue from a lesion, or mass. The concept of flowing parenchyma may relate to mass detection. Dense breast tissue may flow like passing clouds in a series of images whereas, as mass may appear in one or a subset of images. If an area of the volume of tissue flows or changes shape from slice to slice the likelihood of that volume being a mass may be small.

In sound speed images, dense breast tissue flows (e.g., irregularly changes shape or even disappears) from slice to slice as one scrolls through the breast while viewing a stack of images, whereas a lesion, or mass, will remain in an image or uniformly change shape through multiple image slices.

In an example of a qualitative volumetric parameter, a user may indicate whether or not a region of interest “persists” from image type to image type in a plurality of image types. For example, a persist parameter may relate to a mass appearing in more than one ultrasonic image rendering, for example, appearing in both reflection and wafer images. If a mass only appears in one image type, for example, only in a reflection image, the likelihood of that volume of tissue being a real mass may be small. Dark areas in reflection images may generally represent normal tissue. In some cases, if a mass is small in size for example smaller than 1 cm, the mass may not persist between different image types.

In an example of a qualitative volumetric parameter, a user may indicate whether or not a region of interest “stays” on a colorscale image. The parameter “stays” may be used to distinguish dense tissue from a lesion, or mass. In general, it relates to the color staying as the user scrolls through stiffness images in a stiffness image sequence, for example, a sequence that varies by image depth among a series of layers. The color associated with dense tissue may not stay and may pass like a cloud flow as the user scroll through a series of stiffness images. The likelihood of the color staying is high with the cancerous masses.

A set of parameters can include at least 1, 2, 3, 4, 5, 6, 7, 8, 9, 10, 11, 12, 13, 14, 15, 16, 17, 18, 19, 20, 50, 100, 1000, or more parameters.

Other parameters, methods of extraction, and methods of use thereof are described in International Patent Application PCT/US2019/029592, which is incorporated herein by reference in its entirety.

Although the above operations show a method 700 for using a foot controller, in accordance with some embodiments, a person of ordinary skill in the art will recognize many variations based on the teachings described herein. The steps may be completed in any order. Steps may be added or deleted. Some of the steps may comprise sub-steps. Many of the steps may be repeated as often as beneficial to the method.

### Digital Processing Device

In some embodiments, the platforms, systems, media, and methods described herein include a digital processing device, or equivalent, a processor. In further embodiments, the processor includes one or more hardware central processing units (CPUs) or general purpose graphics processing units (GPGPUs) or tensor processing unit (TPU) that carry out the device’s functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the processor includes an operating system configured to perform executable instructions. The operating system is, for example, software, including programs and data, which manages the device’s hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the processor includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the processor is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing-based storage. In further embodiments, the storage and/or memory device is a combination of devices such as those disclosed herein.

In some embodiments, the processor includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the processor, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the processor includes an input device to receive information from a user. The input device is a foot controller. The processor can receive information from multiple sources of input concurrently such as a mouse, keyboard, and foot controller. In some embodiments, input devices in addition to the foot controller can include a pointing device including, by way of non-limiting examples, a mouse, trackball, track pad, joystick, game controller, or stylus. In some embodiments, the input device in addition to the foot controller is a touch screen or a multi-touch screen. In other embodiments, the input device in addition to the foot controller is a microphone to capture voice or other sound input. In other embodiments, the input device in addition to the foot controller is a video camera or other sensor to capture motion or visual input. In further embodiments, the input device in addition to the foot controller is a Kinect, Leap Motion, or the like. In still further embodiments, the input device is a combination of devices such as those disclosed herein.

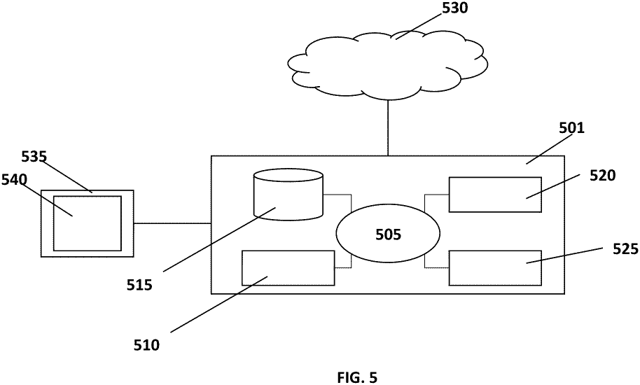

Referring to FIG. 5, in a particular embodiment, an example processor 501 is programmed or otherwise configured to allow presentation of a stack of images of tissue. The processor 501 can regulate various aspects of the present disclosure, such as, for example, input from the foot controller and workstation user interface such as a keyboard and mouse. In this embodiment, the processor 501 includes a central processing unit (CPU, also “processor” and “computer processor” herein) 1205, which can be a single core or multi core processor, or a plurality of processors for parallel processing. The processor 501 also includes memory or memory location 510 (e.g., random-access memory, read-only memory, flash memory), electronic storage unit 515 (e.g., hard disk), communication interface 520 (e.g., network adapter, network interface) for communicating with one or more other systems, and peripheral devices, such as cache, other memory, data storage and/or electronic display adapters. The peripheral devices can include storage device(s) or storage medium 565 which communicate with the rest of the device via a storage interface 570. The memory 510, storage unit 515, interface 520 and peripheral devices are in communication with the CPU 505 through a communication bus 525, such as a motherboard. The storage unit 515 can be a data storage unit (or data repository) for storing data. The processor 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the device 501, can implement a peer-to-peer network, which may enable devices coupled to the device 501 to behave as a client or a server.

Continuing to refer to FIG. 5, the processor 501 includes a foot controller to receive information from a user, the foot controller in communication with other input device(s) via an input interface 550. The processor 501 can include output device(s) 555 that communicates to the foot controller via an output interface 560.

Continuing to refer to FIG. 5, the memory 510 may include various components (e.g., machine readable media) including, but not limited to, a random access memory component (e.g., RAM) (e.g., a static RAM “SRAM”, a dynamic RAM “DRAM, etc.), or a read-only component (e.g., ROM). The memory can also include a basic input/output system (BIOS), including basic routines that help to transfer information between elements within the processor, such as during device start-up, may be stored in the memory 510.

Continuing to refer to FIG. 5, the CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and write back. The CPU 505 can be part of a circuit, such as an integrated circuit. One or more other components of the device 501 can be included in the circuit. In some cases, the circuit is an application specific integrated circuit (ASIC) or a field programmable gate array (FPGA).

Continuing to refer to FIG. 5, the storage unit 515 can store files, such as drivers, libraries and saved programs. The storage unit 515 can store user data, e.g., user preferences and user programs. The processor 501 in some cases can include one or more additional data storage units that are external, such as located on a remote server that is in communication through an intranet or the Internet. The storage unit 515 can also be used to store operating system, application programs, and the like. Optionally, storage unit 515 may be removably interfaced with the processor (e.g., via an external port connector (not shown)) and/or via a storage unit interface. Software may reside, completely or partially, within a computer-readable storage medium within or outside of the storage unit 515. In another example, software may reside, completely or partially, within processor(s) 505.

Continuing to refer to FIG. 5, the processor 501 can communicate with one or more remote computer systems 502 through the network 530. For instance, the device 501 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Continuing to refer to FIG. 5, information and data can be displayed to a user through a display 535.

Methods as described herein can be implemented by way of machine (e.g., computer processor) executable code stored on an electronic storage location of the processor 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

### Non-Transitory Computer Readable Storage Medium

In some embodiments, the platforms, systems, media, and methods disclosed herein include one or more non-transitory computer readable storage media encoded with a program including instructions executable by the operating system of an optionally networked processor. In further embodiments, a computer readable storage medium is a tangible component of a processor. In still further embodiments, a computer readable storage medium is optionally removable from a processor. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the processor’s CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

In some embodiments, the user may be able to scroll within a plurality of images by input through the foot controller. In some cases, the user may be able to toggle between image types to compare the images by input through the foot controller. In some embodiments different image types for example renderings of ultrasound images such as sound speed and reflection may be shown next to each other or in other order for user to make comparisons by input through the foot controller. In some cases, the user may be able to zoom in or zoom out in the images to focus on certain features by input through the foot controller. In some embodiments, different image types may be presented in a certain order by input through the foot controller. In other cases, there may be no order in presentation of different images Web application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

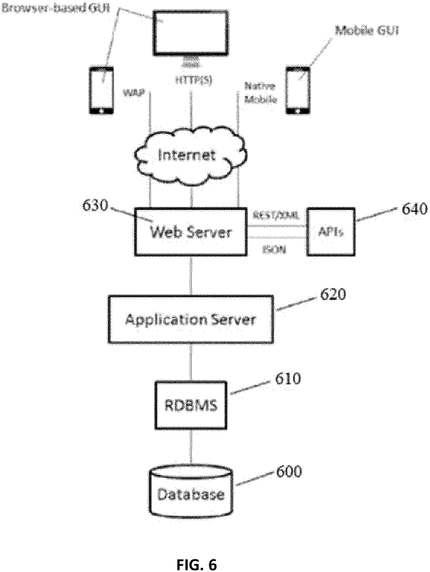

Referring to FIG. 6, in a particular embodiment, an application provision system comprises one or more databases 600 accessed by a relational database management system (RDBMS) 610. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 620 (such as Java servers, .NET servers, PHP servers, and the like) and one or more web servers 630 (such as Apache, IIS, GWS and the like). The web server(s) optionally expose one or more web services via app application programming interfaces (APIs) 640. Via a network, such as the Internet, the system provides browser-based and/or mobile native user interfaces.

In view of the disclosure provided herein, a mobile application can be created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location. The systems and methods disclosed herein may be implemented in the form of a mobile application or a computer software program.

In some embodiments, the platforms, systems, media, and methods disclosed herein include one or more databases, or use of the same. In view of the disclosure provided herein, those of skill in the art will recognize that many databases are suitable for storage and retrieval of raw image data, reconstructed image data, ROIs, stored classified data, label or classification, features, subcategory of features, etc. In various embodiments, suitable databases include, by way of non-limiting examples, relational databases, non-relational databases, object-oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### EXAMPLES

### Example 1: Control and Manipulation of a Breast Tissue Image Stack in SoftVue

A foot controller as described herein is used to control primary and secondary functions of Image Review in a SoftVue Application in review of a breast tissue image. The primary functions include scrolling through images toward a chest wall, scrolling through images toward a nipple and navigating through SoftVue hanging protocols, or image layouts. SoftVue hanging protocol layouts include Bilateral Review, Wafer Sound Speed, Reflection Sound Speed, Wafer Stiffness Fusion, etc as can be seen in FIG. 8. Secondary functions include zooming in an out in an image, centralizing a cursor on the image stacks, auto rotation, 2-D measurements, etc as can be seen in FIG. 9. The primary function of scrolling through a stack of images from chest wall to nipple is controlled by pressing down on a left toe switch. The primary functions of scrolling through a stack of images from nipple to chest wall is controlled by pressing down on a right toe switch. The primary function of moving back through SoftVue image review hanging protocols, or image layouts, is controlled by pressing down on a left heel switch. The primary function of moving forward through SoftVue image review hanging protocols, or image layouts, is controlled by pressing down of a right heel switch. The foot pedal is engaged to zoom in on an image, as the pedal is pressed further down, the image is proportionally zoomed in. Secondary functions are controlled by a hand controller such as a mouse.

### Example 2: Control and Manipulation of Image Characterization Parameters of an Image Stack

A foot controller as described herein, comprising an accelerometer and a gyroscope in a device that clips on to a user’s shoe, is used to control a parameter of a set of parameters of an image of a stack of ultrasound images. The user scrolls through a set of ultrasound reflection images by moving their foot from a neutral position to a first position as is described herein (movement can be in vertical or horizontal plane). The user selects an image of interest by moving their foot to a neutral position. The user taps their foot to access a secondary selecting function and uses a second controller of a user interface such as a keyboard to identify a non-flowing mass in the image of interest. The secondary function can be zoom or selection of hanging protocol / image layout, which are functions that facilitate evaluation of a region of interest detected while scrolling. Dark areas in reflection images may generally represent normal tissue. The non-flowing mass uniformly changes shape through multiple image slices. The user then taps their foot to return to primary controls and moves their foot to a first position to scroll through the remaining series of ultrasound reflection images in the stack of images. The user repeats the previous steps to identify a series of images with the mass and subsequently taps their foot to access a secondary selecting function and uses a second controller of a user interface such as a keyboard to identify the series of images with the non-flowing mass to determine if the mass flows from layer to layer of the stack of ultrasound reflection images.

A foot controller as described herein is used to identify whether or not the second region of interest persists from image type to image type in a plurality of images types. A persist parameter relates to a mass appearing on more than one ultrasonic image rendering, such as in a reflection image and a wafer image. If the mass only appears in one image type, the likelihood of that volume of tissue being a mass is small. A user selects a set of wafer images corresponding to the same tissue as the set of ultrasound reflection images previously reviewed using the user interface. The user scrolls through the set of wafer images by moving their foot from a neutral position to a first position as is described herein. The user selects a wafer image of interest by moving their foot to a neutral position. The user taps their foot to access a secondary hanging protocol or image layout selection function and uses left or right movements on the foot controller to switch between a wafer image layout and a reflection image layout, that correlates to the same section of tissue as a reflection image with a previously identified region of interest.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of manipulating a plurality of tissue images, comprising:
providing an image analysis system, said system comprising:
i. a computing device, including a processor, a memory, and a display;
ii. one or more foot controllers operatively coupled to said computing device, a foot controller of said one or more foot controllers comprising:
a foot motion input receiver capable of detecting vertical movement between a first point and a second point; and
iii. the memory of the computing device storing instructions for the processor to implement a first image manipulation, said first image manipulation being a scrolling function through said plurality of tissue images in response to the foot controller being operated by a user, and cause the display to show the scrolled tissue images.

2. The method of claim 1, wherein said foot controller comprises one or more horizontal motion input receivers, the one or more horizontal motion input receivers being capable of detecting movement in a horizontal plane of motion between an inner point and an outer point, wherein said outer point is located radially away from said inner point.

3. The method of claim 2, wherein said image analysis system further comprises a second image manipulation, said second image manipulation being a selection function for hanging protocols, or different combinations of image layouts and image types.

4. The method of claim 3, further comprising determining a horizontal position of one or more horizontal motion input receivers and controlling a value of said second image manipulation as a function of said position of said one or more horizontal motion input receivers in said horizontal plane of motion.

5. The method of claim 4, wherein said controlling is proportional to the movement of the one or more horizontal motion input receivers as compared to a neutral starting position.

6. The method of claim 1, further comprising proportionally controlling a value of said first image manipulation as a function of said foot motion input receiver in said vertical plane of motion.

7. The method of claim 1, further comprising determining a position of said foot motion input receiver between said first point and said second point.

8. The method of claim 1, wherein said foot controller comprises a heel switch configured to move in a vertical plane of motion between an engaged position wherein said heel switch is level with a stationary heel rest and an unengaged position wherein said heel switch is not level with said stationary heel rest.

9. The method of claim 1, wherein said image analysis system further comprises a second image manipulation, said second image manipulation being an automatic scroll function.

10. The method of claim 9, further comprising determining a position of said heel switch and engaging or disengaging said heel switch.

11. The method of claim 1, wherein said foot controller comprises one or more side switches configured to send a signal to said computer to zoom in or out when engaged.

12. The method of claim 11, wherein said image analysis system further comprises a second image manipulation, said second image manipulation being a zoom function.

13. The method of claim 12, further comprising determining a position of said one or more side switches and proportionally controlling a value of said second image manipulation as a function of engagement of said one or more side switches.

14. The method of claim 1, wherein said first point is correlated with tissue images of a distal part of a tissue and said second point is correlated with tissue images of a proximal part of said tissue.

15. The method of claim 1, wherein said digital stack of tissue images are of a breast tissue.

16. The method of claim 1, wherein said digital stack of tissue images are ultrasound images.

17. The method of claim 1, wherein said foot controller is connected to said computer wirelessly.

18. The method of claim 1, wherein said foot controller is connected to said computer via USB.

19. The method of claim 1, wherein said foot motion input receiver is a foot pedal, wherein said first point is a fully undepressed position of said foot pedal and said second point is a fully depressed position of said foot pedal.

20. The method of claim 1, wherein said movement in said vertical plane of motion has a first region between said first point and an intermediate point and a second region between said intermediate point and said second point.

21. The method of claim 20, wherein said scrolling function is maintained at a constant minimum value of one image per movement to said intermediate point in said first region.

22. The method of claim 20, wherein said scrolling function comprises a variable rate of scrolling proportional to the position of said foot pedal between said intermediate position and said second point in said second region.

23. The method of claim 22, wherein said variable rate of scrolling increases in a linear manner in said second region.

24. A system for manipulating a plurality of tissue images, comprising:
a. a computing device, including a processor, a memory, and a display;
b. a foot controller operatively coupled to said computing device, said foot controller comprising a foot motion input receiver capable of detecting vertical movement between a first point and a second point; and
c. the memory of the computing device storing instructions for the processor to implement a first image manipulation, said first image manipulation being a scrolling function through said plurality of tissue images in response to the foot controller being operated, and cause the display to show the scrolled tissue images.

25. The system of claim 24, wherein said foot controller comprises a heel switch configured to move in a vertical plane of motion between an engaged position wherein said heel switch is level with a stationary heel rest and an unengaged position wherein said heel switch is not level with said stationary heel rest.

26. The system of claim 24, wherein said foot controller comprises one or more side switches comprising one or more dial mode binary switches.

27. The system of claim 26, wherein said one or more dial mode binary switches are configured to be engaged into a first mode and a second mode.

28. The system of claim 27, wherein said first mode is activated by a downward application of pressure on a switch of the one or more side switches and said second mode is activated by a sideways application of pressure on a switch of the one or more side switches.

29. A method of manipulating a plurality of tissue images, comprising:
providing an image analysis system, said system comprising:
i. a computing device, including a processor, a memory, and a display;
ii. one or more foot controllers operatively coupled to said computing device, a foot controller of said one or more foot controllers comprising:
a foot motion input receiver capable of detecting a movement between a first point and a second point and a second point; and
iii. the memory of the computing device storing instructions for the processor to implement a first image manipulation and a second image manipulation, said first image manipulation being a scrolling function through said plurality of tissue images in response to the foot controller being operated by a user, and cause the display to show the scrolled tissue images.

30. The method of claim 29, wherein said foot controller comprises one or more horizontal motion input receivers, the one or more horizontal motion input receivers being capable of detecting movement in a horizontal plane of motion between an inner point and an outer point, wherein said outer point is located radially away from said inner point.

31. The method of claim 30, wherein said second image manipulation is a selection function for hanging protocols, or different combinations of image layouts and image types.

32. The method of claim 31, further comprising determining a horizontal position of one or more horizontal motion input receivers and controlling a value of said second image manipulation as a function of said position of said one or more horizontal motion input receivers in said horizontal plane of motion.

33. The method of claim 32, wherein said controlling is proportional to the movement of the one or more horizontal motion input receivers as compared to a neutral starting position.

34. The method of claim 29, wherein said foot motion input receiver is capable of detecting movement in a vertical plane of motion between said first point and said second point.

35. The method of claim 34, further comprising proportionally controlling a value of said first image manipulation as a function of said foot motion input receiver in said vertical plane of motion.

36. The method of claim 29, further comprising determining a position of said foot motion input receiver between said first point and said second point.

37. The method of claim 29, wherein said foot controller comprises a heel switch configured to move in a vertical plane of motion between an engaged position wherein said heel switch is level with a stationary heel rest and an unengaged position wherein said heel switch is not level with said stationary heel rest.

38. The method of claim 34, wherein said second image manipulation is an automatic scroll function.

39. The method of claim 34, further comprising determining a position of said heel switch and engaging or disengaging said heel switch.

40. The method of claim 29, wherein said foot controller comprises one or more side switches configured to send a signal to said computer to zoom in or out when engaged.

41. The method of claim 40, wherein said second image manipulation is a zoom function.

42. The method of claim 41, further comprising determining a position of said one or more side switches and proportionally controlling a value of said second image manipulation as a function of engagement of said one or more side switches.

43. The method of claim 29, wherein said first point is correlated with tissue images of a distal part of a tissue and said second point is correlated with tissue images of a proximal part of said tissue.

44. The method of claim 29, wherein said digital stack of tissue images are of a breast tissue.

45. The method of claim 29, wherein said digital stack of tissue images are ultrasound images.

46. The method of claim 29, wherein said foot controller is connected to said computer wirelessly.

47. The method of claim 29, wherein said foot controller is connected to said computer via USB.

48. The method of claim 29, wherein said foot motion input receiver is a foot pedal, wherein said first point is a fully undepressed position of said foot pedal and said second point is a fully depressed position of said foot pedal.

49. The method of claim 29, wherein said movement in said vertical plane of motion has a first region between said first point and an intermediate point and a second region between said intermediate point and said second point.

50. The method of claim 49, wherein said scrolling function is maintained at a constant minimum value of one image per movement to said intermediate point in said first region.

51. The method of claim 49, wherein said scrolling function comprises a variable rate of scrolling proportional to the position of said foot pedal between said intermediate position and said second point in said second region.

52. The method of claim 51, wherein said variable rate of scrolling increases in a linear manner in said second region.

53. A system for manipulating a plurality of tissue images, comprising:
a. a computing device, including a processor, a memory, and a display;
b. a foot controller operatively coupled to said computing device, said foot controller comprising a foot motion input receiver capable of detecting movement between a first point and a second point; and
c. the memory of the computing device storing instructions for the processor to implement a first image manipulation and a second image manipulation, said first image manipulation being a scrolling function through said plurality of tissue images in response to the foot controller being operated, and cause the display to show the scrolled tissue images.

54. The system of claim 53, wherein the foot motion input receiver is capable of movement in a vertical plane of motion between said first point and said second point.

55. The system of claim 53, wherein said foot controller comprises a heel switch configured to move in a vertical plane of motion between an engaged position wherein said heel switch is level with a stationary heel rest and an unengaged position wherein said heel switch is not level with said stationary heel rest.

56. The system of claim 53, wherein said foot controller comprises one or more side switches comprising one or more dial mode binary switches.

57. The system of claim 56, wherein said one or more dial mode binary switches are configured to be engaged into a first mode and a second mode.

58. The system of claim 57, wherein said first mode is activated by a downward application of pressure on a switch of the one or more side switches and said second mode is activated by a sideways application of pressure on a switch of the one or more side switches.

59. The system of claim 57, wherein said second image manipulation is a zoom function.

60. The system of claim 57, wherein said second image manipulation is an automatic scroll function.
